(19) United States
(12) Patent Application Publication
(10) Pub. No.: US 2025/0121024 A1
(43) Pub. Date: Apr. 17, 2025

(54) COMBINED USE OF IMIQUIMOD AND A CASEIN HYDROLYSATE FOR THE TREATMENT OF CONDYLOMA

(71) Applicant: NTD LABS, S.L.U., BARCELONA (ES)

(72) Inventor: GUILLERMO LOPEZ ZARCO, BARCELONA (ES)

(73) Assignee: NTD LABS, S.L.U., BARCELONA (ES)

(21) Appl. No.: 18/701,834

(22) PCT Filed: Nov. 4, 2022
(86) PCT No.: PCT/IB2022/060635

§ 371 Date: Apr. 16, 2024

(30) Foreign Application Priority Data
Nov. 9, 2021 (ES) P202131044

(51) Int. Cl. A61K 38/01; A61K 9/00; A61K 31/4745; A61P 17/00; A61P 31/20

(52) U.S. Cl. CPC A61K 38/018; A61K 9/0014; A61K 9/0056; A61K 31/4745; A61P 17/00; A61P 31/20

(57) ABSTRACT

The present invention relates to the combined use of imiquimod and a casein hydrolysate for the treatment of condyloma. The composition of the casein hydrolysate is determined by the use of a proline-specific endoprotease for the hydrolysis of casein and is characterized in that the molar fraction of the peptides of said hydrolysate carrying a carboxy terminal proline, expressed in %, is more than two times the molar fraction of proline, expressed in %, in the casein substrate used to generate the hydrolysate. By means of this combination, a complete remission of the warts and the absence of recurrences are achieved in patients suffering from condyloma. The present invention also relates to a pharmaceutical composition for topical administration comprising the combination of imiquimod and a casein hydrolysate.

### TECHNICAL FIELD

The present invention relates to the treatment of genital warts (condyloma), more specifically, it relates to the combined use of imiquimod and a casein hydrolysate for the treatment of condyloma.

### STATE OF ART

One of the most frequent clinical manifestations of human papillomavirus (HPV) infection are genital or anogenital warts, also known as condyloma acuminata or simply condyloma. They are fleshy papules, usually skin-coloured, that appear in the anogenital region.

Among the more than 100 different types of HPVs identified so far, types 6 and 11 are considered to be responsible for the majority of condyloma. HPV infections are considered a sexually transmitted viral disease and, in adults, condyloma is generally exclusively transmitted through sexual contact.

The usual therapeutic approach to condyloma includes various topical pharmacological treatments, among which podophyllotoxin, sinecatechins and imiquimod are the most common. In the article Jung et al., Topically applied treatments for external genital warts in nonimmunocompromised patients: a systematic review and network meta-analysis, Br. J. Dermatol., 2020, 183, 24-36, the various available alternatives are comparatively reviewed and it is concluded that, for example, a 0.5% podophyllotoxin solution is significantly more effective than a 5% imiquimod cream for wart removal, but it has the disadvantage of being associated with higher proportion of adverse effects; on the other hand, a 15% sinecatechin lotion is significantly less effective than 5% imiquimod; additionally, it was found that imiquimod formulations at 2.5% or 3.5% show significantly less efficacy than one at 5%. On the other hand, it was determined that the recurrence rates were similar for all treatments.

There are also other non-pharmacological alternatives for the elimination of condyloma, for example, cryotherapy, through the application of liquid nitrogen. Likewise, there is the option of the surgical resection of the wart, according to various alternatives, including electrocautery and laser therapy. Destructive chemical methods can also be used, for example, with trichloroacetic or bichloroacetic acids, although such treatments cause irritation and are not uniformly effective.

So far, surgical treatments are the only ones that can provide virtually 100% remission rates, although they have the disadvantage of being aggressive treatments and that they can lead to the appearance of scars and recurrence. Thus, despite the treatments suggested so far, surgical removal of condyloma remains the main treatment of choice (Mistrangelo et al., Minerva Chir., 2018, 73 (1), 100-106).

Regardless of the treatment selected, it is estimated that the overall recurrence rate is usually at least 20-30%, with the majority of recurrences being observed between three and six months after completion of the treatment, while after 1-2 years the risk of recurrences decreases. In general, it is recommended to treat recurrent condyloma with the same primary treatments that were effective (AEPCC-Guía, “Condilomas acuminados”, AEPCC Publications, November 2015).

Therefore, there is still a need to have an effective treatment for the elimination of condyloma, which is capable of providing a complete remission of the warts, as well as preventing their recurrence, which is non-invasive, and with which side effects and possible consequences derived from treatment are minimized.

### OBJECT OF THE INVENTION

The object of the present invention is the combination of imiquimod and a casein hydrolysate for use in the treatment of condyloma.

Another aspect of the invention is a pharmaceutical composition for topical administration comprising the combination of imiquimod and a casein hydrolysate.

### DETAILED DESCRIPTION OF THE INVENTION

The object of the present invention is the combination of imiquimod and a casein hydrolysate for use in the treatment of condyloma, wherein said hydrolysate comprises peptides wherein the molar fraction of peptides carrying a carboxy terminal proline, expressed in %, is more than two times the molar fraction of proline, expressed in %, in the casein substrate used to generate the hydrolysate.

The authors of the present invention have developed a new therapy for the treatment of condyloma, based on the combination of imiquimod and a specific casein hydrolysate, which provides greater efficacy in the elimination of condyloma compared to pharmacological therapies available so far, achieving complete remission of warts and absence of recurrences in the patients suffering from condyloma.

### Definitions

Along the present description, as well as in the claims, expressions in the singular, generally preceded by the articles “a”, “an”, or “the”, are intended to include plural forms as well, unless the context clearly indicates otherwise. Furthermore, numerical values preceded by the terms “approximately” or “about” are intended to include the exact indicated value and also some variation around said value, in particular, a variation of ±5% around the indicated amount. The numerical ranges, defined by the lower and upper limits thereof, are understood to also include the values of said limits.

Along the present description, as well as in the claims, the percentages are generally by weight (% w, % w/w), unless the context clearly indicates otherwise. Thus, for example, the percentage of the various components of the described formulations refers to their proportion by weight relative to the total weight of the composition. However, the percentages that define the peptide composition of the casein hydrolysate clearly refer to the molar fraction, and in this specific case it is clearly understood that they are not percentages by weight.

### Condyloma

Condyloma, as is well known to those skilled in the art, are benign anogenital warts caused by the human papillomavirus (HPV). Along the present description, the terms condyloma, condyloma acuminata, genital warts, venereal warts, or anogenital warts may be used interchangeably.

As is well known, most condylomas are caused by HPV genotypes 6 and 11, although, less frequently, other genotypes may also be involved in the appearance of condyloma (8, 13, 30, 32, 42, 43, 44, 54, 55, 70). Condyloma is transmitted mainly by sexual contact, with a high rate of transmissibility. The main risk factors for contracting condyloma are the number of sexual partners and an early initiation of sexual intercourse. Likewise, it is a particularly frequent pathology in immunocompromised patients. (AEPCC-Guía, “Condilomas acuminados”, AEPCC Publications, November 2015).

Condyloma often occur as clusters, which may be very small or may spread in large masses over the genital or anal area.

In general, warts are asymptomatic, but in some patients, they cause itching, burning or discomfort. In men, warts occur most commonly under the foreskin, on the coronal sulcus, within the urethral meatus, and on the penile shaft, although they may also occur around the anus and in the rectum. In women, warts occur most commonly on the vulva, vaginal wall, cervix, and perineum, although the urethra and anal region may also be affected (Merck Manual, Professional Version, https://www.merckmanuals.com/professional).

The diagnosis of condyloma is well established, as described, for example, in the AEPCC Guide, cited above, and includes techniques such as physical examination, biopsy and molecular diagnosis.

### Treatment of Condyloma

In the context of the present invention, the term “treatment” of condyloma refers to the use of the combination of the invention for curative purposes, once the onset of anogenital warts (condyloma) has been diagnosed. By cure is meant either the complete disappearance of the lesion, or its mitigation or improvement, for example, the decrease in the number of condyloma or the lesion area present.

The treatment of condyloma also refers to the prevention of recurrence once the condyloma disappears. Recurrence is frequent in these lesions caused by the human papillomavirus since it can remain in an asymptomatic latent state in the infected individual for prolonged periods.

In the context of the present invention, the treatment of condyloma is limited to the treatment of humans.

### Imiquimod

Imiquimod (1-(2-methylpropyl)-1H-imidazo[4,5-c]quinoline-4-amine, CAS number 99011-02-6) is an immunomodulator drug that increases the local immune response mediated by interferon and other cytokines.

Imiquimod is a well-known substance, which can be prepared by known methods, described in the literature, for example, as described in the book D. Lednicer, The Organic Chemistry of Drug Synthesis, Volume 5, John Wiley & Sons, 1995, 170-181, or as described in U.S. Pat. No. 4,689,338. Furthermore, imiquimod is widely commercially available from various suppliers.

Imiquimod, administered topically, is indicated for the treatment of condyloma. Thus, for example, the drug Aldara® is a 5% imiquimod cream authorized by the European Medicines Agency for the treatment of condyloma acuminata. The suggested posology is three times per week, until the clearance of visible genital or perianal warts or for a maximum of 16 weeks per episode of warts.

### Casein Hydrolysate

The casein hydrolysate is a specific hydrolysate that is characterized in that the molar fraction of the peptides of said hydrolysate carrying a carboxy terminal proline, expressed in %, is more than two times the molar fraction of proline, expressed in %, in the casein substrate used to generate the hydrolysate.

As is well known, casein, the origin of the hydrolysate of the invention, encompasses a group of phosphoproteins present in milk and constitutes approximately 3% of bovine milk. The main components of casein are alpha-, beta-, gamma- and kappa-caseins, of which beta casein is the major fraction of bovine milk.

Casein used as the substrate of the hydrolysis is preferably bovine milk casein, more preferably bovine milk beta-casein.

The casein hydrolysate used in the present invention, as defined above, is a characteristic hydrolysate whose composition is determined by the use of a proline-specific endoprotease in the hydrolysis of casein, so that the hydrolysate has a composition characterized by a high content of peptides with a proline at the carboxy-terminal end.

The characteristics of said hydrolysate and of the proline-specific endopeptidase used in its preparation are described in international patent application WO-A-02/45524. Said document also describes the method to determine the molar fraction of peptides carrying a carboxy terminal proline, expressed in %, as well as the method to determine the molar fraction of proline, expressed in %, in the casein substrate used to generate the hydrolysate.

Preferably, the molar fraction of peptides in said hydrolysate carrying a carboxy terminal proline, expressed in %, is at least three times the molar fraction of proline, expressed in %, in the casein substrate used to generate the hydrolysate.

Preferably, the casein hydrolysate according to the use of the present invention is characterized in that the average length of the peptides in the hydrolysate is comprised between 3 and 9 amino acids.

Preferably, the molar fraction of peptides carrying a carboxy terminal proline in the casein hydrolysate is at least 25%, and still more preferably is comprised between 30% and 70%.

In the context of the present invention, when discussing the molar fraction of peptides, peptides are understood to be those whose molecular mass is comprised between 400 and 2000 Daltons, which can be determined, for example, by the liquid chromatography/mass spectrometry (LC/MS) method described in the Materials and Methods section of the above cited international patent application WO-A-02/45524.

In general, the casein hydrolysate according to the use of the present invention is a hydrolysate where at least 50% of the casein substrate is hydrolysed. Preferably, at least 10% of the casein substrate is converted into peptides of molecular mass comprised between 400 and 2000 Daltons; more preferably, between 20% and 90%, and still more preferably, between 30% and 80% of the casein substrate is converted into such peptides.

In a preferred embodiment, the casein hydrolysate has the following amino acid composition: between 54-64 of lysine, between 22-30 of methionine, between 25-33 of threonine, between 18-26 of histidine, between 26-34 of arginine, between 46-56 of valine, between 34-42 of isoleucine, between 70-80 of leucine, between 33-41 of phenylalanine, between 45-55 of the sum of aspartic acid and asparagine, between 160-180 of the sum of glutamine and glutamic acid, between 20-26 of alanine, between 80-90 of proline, between 40-48 of tyrosine, between 37-45 of serine, between 11-17 of glycine, between 0.8-1.2 of cysteine, and between 6-10 of tryptophan; wherein the amounts are expressed in grams of each amino acid per kilogram of hydrolysate.

In a more preferred embodiment, the casein hydrolysate has the following amino acid composition: between 57-61 of lysine, between 24-28 of methionine, between 27-31 of threonine, between 20-24 of histidine, between 28-32 of arginine, between 49-53 of valine, between 36-40 of isoleucine, between 73-77 of leucine, between 35-39 of phenylalanine, between 48-52 of the sum of aspartic acid and asparagine, between 166-176 of the sum of glutamine and glutamic acid, between 21-25 of alanine, between 82-88 of proline, between 42-46 of tyrosine, between 39-43 of serine, between 13-15 of glycine, between 0.8-1.2 of cysteine, and between 7-9 of tryptophan; wherein the amounts are expressed in grams of each amino acid per kilogram of hydrolysate.

In a particularly preferred embodiment, the casein hydrolysate has approximately the following amino acid composition: 59 of lysine, 26 of methionine, 29 of threonine, 22 of histidine, 30 of arginine, 51 of valine, 38 of isoleucine, 75 of leucine, 37 of phenylalanine, 50 of the sum of aspartic acid and asparagine, 171 of the sum of glutamine and glutamic acid, 23 of alanine, 85 of proline, 44 of tyrosine, 41 of serine, 14 of glycine, 1 of cysteine, and 8 of tryptophan; wherein 35 the amounts are expressed in grams of each amino acid per kilogram of hydrolysate.

International patent application WO-A-2015/125067 describes the use of the casein hydrolysate of the invention for the prevention and treatment of various viral infections, including those caused by the human papilloma virus. Example 7 describes the oral use of said hydrolysate in four male patients affected by condyloma.

A casein hydrolysate according to the characteristics described above is commercially available under the name PeptoPro® (DSM), to enrich the protein content of foods and beverages.

Furthermore, a casein hydrolysate according to these characteristics is also commercially available under the name HuPaVir®, for the treatment and prevention of HPV infections, including the treatment of condyloma.

### Combined use of Imiquimod and the Casein Hydrolysate

As described in Example 3, the authors of the present invention have observed that the treatment of condyloma with the combination of imiquimod and the casein hydrolysate provides excellent results, with complete resolution of condyloma in 100% of patients, already after 12 weeks of treatment. This is in contrast to the results obtained with the individual treatments, with which, with this same treatment time, total remission is only achieved in 58% (imiquimod) or 50% (casein hydrolysate) of treated patients. Furthermore, none of the patients treated with the combination experienced recurrences after 6 months from the start of treatment.

These results are surprising, as well as encouraging in terms of having an effective, safe and minimally invasive treatment for anogenital warts, since, so far, the only treatment capable of achieving 100% of total remission is surgery.

Furthermore, due to the fact that casein hydrolysate is a totally innocuous product, derived from the hydrolysis of casein from bovine milk and even commonly used as a dietary supplement, the use according to the present invention has the advantage that it is possible to significantly increase the efficacy of imiquimod without added side effects, as might possibly occur with the added use of other antivirals. In addition, this allows, if considered appropriate for the prevention of recurrence, to maintain the treatment with the casein hydrolysate alone for prolonged periods of time in a completely safe manner, once the remission of condyloma has been achieved with the combination of the invention.

The combined use of imiquimod and the casein hydrolysate for the treatment of condyloma, according to the present invention, can generally be prolonged for a maximum period of about 6 months for each episode of condyloma treated, or the treatment can be interrupted before, once the condyloma has already disappeared. Preferably, the period of joint treatment is a maximum of about 5 months, more preferably a maximum of about 4 months, and still more preferably a maximum of about 16 weeks, for each episode of condyloma treated, according to the recommendations generally applicable for topical imiquimod.

Optionally, once the joint treatment with imiquimod and casein hydrolysate has ended, either because the maximum recommended period for imiquimod has been reached or because the warts have disappeared, the administration of the casein hydrolysate can be continued on its own, without being combined with imiquimod. for an additional period of time that can be comprised between a few weeks and several months, for example, for from 1 to 10 additional weeks, or from 1 to 12 additional months. As indicated above, casein hydrolysate is a product of natural origin, completely harmless, that does not cause unwanted side effects, and its subsequent prolonged use can contribute to the prevention of condyloma recurrence.

According to the use of the present invention, imiquimod is typically administered topically.

To this end, an imiquimod pharmaceutical composition suitable for topical administration is used, for example, in the form of a cream or ointment, among other possible options, with an imiquimod content generally comprised between 2% and 8%, preferably comprised between 3% and 5%, for example, of about 5%.

The posology of topically administered imiquimod can be adjusted to each particular case. For example, it can be administered once per day, or every other day, or 1 time per week, or 2 times per week, or 3 times per week, or 4 times per week, or 5 times per week. A preferred posology is 3 times per week.

The imiquimod topical composition is administered in the usual way, as is already known for the authorized commercial forms of this drug. Typically, a thin layer of the composition is administered, which is spread over the surface of the wart and, preferably, after each administration, care should be taken that the product remains in contact with the warts for a sufficient period of time, typically, between 6 and 10 hours, so it is recommended to apply the product prior to normal sleeping hours.

In an embodiment of the invention, imiquimod is administered topically and the casein hydrolysate is administered orally.

In another embodiment of the invention, imiquimod is administered topically and the casein hydrolysate is also administered topically and, optionally, the casein hydrolysate is also simultaneously administered orally.

According to the combined use of the present invention, when the casein hydrolysate is administered orally, the daily oral dose used is typically comprised between 1 g and 30 g, preferably comprised between 2 g and 20 g, more preferably comprised between 3 g and 15 g, and still more preferably comprised between 4 g and 10 g, for example, the dose can be selected from about 4, 5, 6, 7, 8, 9 and 10 grams of hydrolysate per day. Said daily dose can be divided into from 1 to 3 doses per day, preferably it is administered in a single dose per day. A particularly preferred posology is a daily dose of about 6 g of casein hydrolysate taken in one single administration.

When casein hydrolysate is administered topically, like imiquimod, one option is to administer both substances separately, that is, using two independent topical formulations. In this way, the posology can be adapted in a more versatile way to the specific needs of each patient. For example, the posology of imiquimod can be selected from once per day, or every other day, or 1 time per week, or 2 times per week, or 3 times per week, or 4 times per week, or 5 times per week; and the posology of the casein hydrolysate can be selected, for example, from once per day, or every other day, or 1 time per week, or 2 times per week, or 3 times per week, or 4 times per week, or 5 times per week. A possible option is to alternate both treatments, on alternate days, but any other combination can be valid. A person skilled in the art will have no difficulty in designing a suitable posology combining both products. Typically, the posology will be adjusted so that the recommended maximum administration for imiquimod is not exceeded, so a preferred posology is to administer imiquimod 3 times per week, distributed throughout the week, and the casein hydrolysate the other days, up to a total of 4 times per week.

Typically, a pharmaceutical composition of the casein hydrolysate suitable for topical administration is used, for example, in the form of a cream or ointment, among other possible options, with a content of the casein hydrolysate generally comprised between 5% and 25%, preferably comprised between 7% and 15%.

When the casein hydrolysate is administered topically, another option is that imiquimod and the casein hydrolysate are combined in a single topical formulation, for example, in the form of a cream or ointment, comprising both substances. This option may be more convenient for the patient, in order to reduce the number of applications of the product and thus also favour a better therapeutic compliance. The posology of the combined topical product can be the same described above for imiquimod, for example, once per day, or every other day, or 1 time per week, or 2 times per week, or 3 times per week, o 4 times per week, o 5 times per week, preferably 3 times per week.

Typically, a pharmaceutical composition suitable for topical administration simultaneously comprising imiquimod and the casein hydrolysate is used, for example, in the form of a cream or ointment, among other possible options, with a content of imiquimod generally comprised between 2% and 8%, preferably between 3% and 5%, and more preferably of about 5%, and a content of the casein hydrolysate comprised between 5% and 25%, more preferably comprised between 7% and 15%.

Additionally, in addition to administering both substances topically, the treatment can be complemented with the administration of the casein hydrolysate also orally, according to the same posology described above.

As indicated above, optionally, once the joint treatment with imiquimod and the casein hydrolysate has ended, either if the latter is administered orally, or topically or both, the administration of the casein hydrolysate can be continued on its own, without being combined with imiquimod, for an additional period of time which may be comprised between a few weeks and several months, for example, for between 1 and 10 additional weeks, or between 1 and 12 additional months. During this additional period, the casein hydrolysate can be used orally and/or topically, preferably orally.

The combination of imiquimod and casein hydrolysate, according to the use of the present invention, can optionally be supplemented with other known substances that favour the strengthening of the immune system and that can, therefore, contribute to the elimination of condyloma caused by HPV. For example, during treatment, the intake of vitamins such as A, B₆, B₁₂, C, D, E, and folate, micronutrients such as zinc, iron, copper, magnesium, and selenium, or omega-3 fatty acids such as eicosapentaenoic and docosahexaenoic acid may be increased, either using suitable dietary supplements or, optionally, combining said nutrients in oral formulations of the casein hydrolysate.

### Pharmaceutical Compositions

As described above, in the combined use according to the present invention, imiquimod is typically administered topically while casein hydrolysate may be administered orally or topically.

To this end, both active ingredients are formulated in the most convenient form for each specific therapeutic regimen.

The pharmaceutical composition or compositions suitable for the use according to the present invention are all those suitable for oral or topical administration and are generally conventional compositions, which can be prepared using standard methods, well known to those skilled in the art, such as those described in pharmaceutical technology manuals, such as, for example, Remington The Science and Practice of Pharmacy, 20^{th} Edition, Lippincott, Williams & Wilkins, Philadelphia, 2000 [ISBN: 0-683-306472], or in M. E. Aulton and K. M. G. Taylor, Aulton's Pharmaceutics, the design and manufacture of medicines, 4^{th} edition, Churchill Livingstone Elsevier, 2013 [ISBN: 978-0-7020-4290-4], among others.

The excipients suitable to be used in the pharmaceutical compositions of the present invention are also well known to those skilled in pharmaceutical technology and are described, for example, in the book R. C. Rowe, P. J. Sheskey and P. J. Weller, Handbook of Pharmaceutical Excipients, Fourth Edition, Pharmaceutical Press, 2003.

Imiquimod is typically formulated as a composition for topical use. In an embodiment of the invention, the casein hydrolysate is also formulated as a separate topical composition. In another embodiment of the invention, imiquimod and casein hydrolysate are formulated in a single combined topical formulation.

Any pharmaceutical form suitable for topical administration can be used, whether in solid, liquid or semisolid form. Solid compositions for topical administration are generally in powder form, and may include a suitable carrier, such as, for example, talc, silica, or microcrystalline cellulose, among others. Liquid compositions suitable for topical administration can be prepared by dissolving or dispersing the active ingredient(s) in a suitable vehicle such as, for example, water, alcohols, glycols, or mixtures thereof, and are, for example, lotions, liniments, or tinctures; or else this liquid composition can be used to impregnate a support, in the form of dressing or bandage that is applied to the affected area; or alternatively the liquid composition can be sprayed onto the affected area using pump or aerosol sprays. Other forms of topical administration are semisolid compositions, such as creams, gels, ointments, or pastes.

Preferably, compositions are used in the form of a cream, gel, ointment or paste, more preferably in the form of a cream or ointment.

Some illustrative examples of the excipients and vehicles commonly used to prepare liquid or semisolid topical formulations are: solvents such as water, alcohol, almond oil, castor oil, rapeseed oil, glycerin, among others; buffers such as diethanolamine, dibasic sodium phosphate, monobasic sodium phosphate, potassium citrate, sodium bicarbonate, sodium citrate dihydrate, among others, and mixtures thereof; viscosity modifiers such as alginic acid, bentonite, carbomers, carrageenan, gelatin, glycerin, hydroxyethylcellulose, hydroxyethylmethylcellulose, hydroxypropylcellulose, maltodextrin, polyvinyl alcohol, sodium alginate, tragacanth, gum arabic, or xanthan gum, among others, and mixtures thereof; emulsifiers such as calcium stearate, cetyl alcohol, ethylene glycol palmitostearate, glycerin monostearate, lecithin, oleic acid, poloxamers, sodium lauryl sulfate, polysorbates, sorbitan esters, polyethoxylated castor oil derivatives, or emulsifying wax, among others, and mixtures thereof; suspending agents such as xanthan gum, guar gum, alginic acid, bentonite, carbomers, sodium or calcium carboxymethylcellulose, hydroxyethylcellulose, hydroxypropylcellulose, hydroxypropylmethylcellulose, hydroxypropyl alginate, microcrystalline or powdered cellulose, anhydrous colloidal silica, dextrins, gelatins, kaolin, magnesium aluminum silicate, maltitol, povidone, sorbitan esters, or tragacanth, among others, and mixtures thereof; humectants such as benzalkonium chloride, sodium docusate, sodium lauryl sulfate, sorbitan esters, polyethoxylated stearates, or polyethoxylated sorbitan fatty esters, among others, and mixtures thereof; emollients such as almond oil, castor oil, coconut oil, fatty esters of glycerin or isopropyl alcohol, cetyl alcohol, cetostearyl alcohol, stearyl alcohol, oleyl alcohol, lecithin, lanolin, or medium chain triglycerides, among others, and mixtures thereof; preservatives such as benzalkonium chloride, benzyl alcohol, bronopol, parabens, sodium benzoate, sodium propionate, sorbic acid, or thimerosal, among others, and mixtures thereof; or mixtures of the above.

The skilled in the art will have no difficulty in selecting and combining the most suitable excipients to prepare topical formulations that comprise imiquimod, or the casein hydrolysate, or a combination of both as active ingredients.

The semisolid topical formulations in the form of creams, gels, ointments or pastes, for example, typically comprise a pharmaceutically acceptable vehicle in which the active ingredient is dissolved, emulsified, dispersed or suspended. Said vehicle can be water, a non-aqueous vehicle miscible in water, such as ethanol or isopropanol, for example, or a non-aqueous vehicle non-miscible in water, such as paraffin oil, for example. Optionally, said semisolid compositions for topical administration contain at least one pharmaceutically acceptable excipient such as, for example, surface-active and emulsifying agents, lipidic compounds and emollients, consistency factors and thickening agents, stabilizers, hydrotropes, preservative agents, essences, colorants, silicone compounds, fats, waxes, lecithins and phospholipids, or mixtures thereof.

Creams, as is well known to those skilled in pharmaceutical technology, are semisolid emulsions, which can be of the oil-in-water (o/w) type or of the water-in-oil (w/o) type, formulated from an oily phase, an aqueous phase and one or more emulsifying agents. The oily phase is made up of a vehicle that can be, for example, liquid paraffin, or a vegetable oil such as, for example, castor, almond, peanut, sesame, cottonseed, olive, rapeseed, soybean, safflower or corn oil.

Gels are obtained from a liquid that is gelled by the addition of a rheological agent or gelling agent. Some gelling agents suitable to be used in the present invention are, for example, carrageenan, guar gum, gum tragacanth, locust bean gum, pectin, agar, alginic acid, carbomers, carboxymethylcellulose, methylcellulose, hydroxyethylcellulose, hydroxypropylmethylcellulose, and polyethylene glycol, among others.

Ointments are fatty semisolid preparations that contain the active ingredient dissolved or in the form of a dispersion. Ointments can be formulated with various vehicles such as paraffin, plastibases (a mixture of polyethylene with a series of hydrocarbons), vegetable oils, such as, for example, peanut, sesame, olive, cottonseed, almond, or corn oil, silicones or polyethylene glycols, among others, or with a mixture of the above.

Pastes are prepared in a similar way to ointments, and have a more solid consistency due to the fact that they contain a greater amount of insoluble solid substances.

When the formulation comprises a vegetable oil, an oil rich in omega-3 fatty acids can be advantageously used, such as rapeseed, olive or soybean oil, for example, due to its known immune-stimulating activity.

The amount of active ingredient in those topical formulations is variable, depending on the type of formulation. For example, for a topical imiquimod composition, for example, in the form of a cream, gel, ointment, or paste, the amount of imiquimod is typically comprised between 2% and 8%, preferably comprised between 3% and 5%. A preferred composition has a 5% of imiquimod.

Topical imiquimod compositions suitable for use in combination with a casein hydrolysate according to the present invention are described, for example, in European patent application EP-A-0376534. This patent application describes the preparation of imiquimod compositions for topical use comprising a fatty acid, for example, isostearic acid or oleic acid, to favour the penetration of the active ingredient.

Additionally, a commercially available topical imiquimod composition can be used according to the use of the present invention, for example, Aldara® 5% cream or other equivalent medicines.

For a topical composition of the casein hydrolysate, for example, in the form of a cream, gel, ointment, or paste, the amount of hydrolysate is typically comprised between 5% and 25%, and more preferably comprised between 7% and 15%.

In an embodiment of the invention, imiquimod and the casein hydrolysate are both administered topically in a single combined formulation. This allows a more convenient administration of both substances and a better therapeutic compliance.

Another aspect of the present invention is a pharmaceutical composition for topical administration comprising imiquimod and a casein hydrolysate as active ingredients and at least one pharmaceutically acceptable excipient, wherein said hydrolysate comprises peptides wherein the molar fraction of peptides carrying a carboxy terminal proline, expressed in %, is more than two times the molar fraction of proline, expressed in %, in the casein substrate used to generate the hydrolysate.

In a preferred embodiment, the composition is selected from cream, gel, ointment and paste, preferably from a cream and an ointment.

Preferably, the combined topical composition comprises between 2% and 8% imiquimod, preferably between 3% and 5%, and more preferably about 5% imiquimod and between 5% and 25% of the casein hydrolysate, more preferably between 7% and 15%.

In an embodiment of the invention, the casein hydrolysate is administered orally to the patients. Said hydrolysate can be administered directly, without being combined with other additives, as a powdery solid; or, preferably, it can be combined with at least one pharmaceutically acceptable excipient and/or vehicle, in the form of a pharmaceutical composition.

Any type of pharmaceutical composition suitable for oral administration is appropriate. As the preferred doses to be administered are relatively high, of several grams, preferred are solid compositions in the form of powder or granules, or liquid compositions, in the form of solution, suspension, or syrup, for example, although other forms for oral administration, as capsules or tablets, can also be used. Preferably, the casein hydrolysate for oral administration of the invention is formulated in powder or granule form.

The pharmaceutically acceptable excipients that can be used for the preparation of oral pharmaceutical compositions in solid form are well known to those skilled in the art. Some illustrative examples are the following: diluents, such as calcium carbonate, sodium carbonate, magnesium carbonate, magnesium oxide, calcium sulfate, calcium phosphate, sodium chloride, microcrystalline or powdered cellulose, cellulose acetate, ethylcellulose, dextrates, dextrins, dextrose, lactose, lactitol, fructose, sorbitol, sucrose, maltodextrins, maltose, glycerin palmitostearate, kaolin, polymethacrylates, pregelatinized starch or starch, among others, and mixtures thereof; lubricating agents such as calcium stearate, magnesium stearate, talc, stearic acid, glycerin behenate, glycerin palmitostearate, sodium benzoate, sodium lauryl sulfate, sodium stearyl fumarate, or hydrogenated castor oil, among others, and mixtures thereof; disintegrants such as alginic acid, croscarmellose sodium, crospovidone, sodium starch glycolate, starch, low-substituted hydroxypropylcellulose, among others, and mixtures thereof; binders such as sodium carboxymethylcellulose, cellulose acetate phthalate, dextrates, dextrin, ethylcellulose, guar gum, maltodextrin, methylcellulose, microcrystalline cellulose, povidone, pregelatinized starch, stearic acid, or sucrose, among others, and mixtures thereof; anti-caking agents such as tribasic calcium phosphate, calcium silicate, colloidal silica, magnesium silicate, magnesium trisilicate, or talc, among others, and mixtures thereof; such as colloidal thickening agents silica, dextrin, ethylcellulose, hydroxyethylcellulose, hydroxyethylmethylcellulose, hypromellose, polyethylene glycol, trehalose, xanthan gum, among others, and mixtures thereof; suspending agents such as xanthan gum, guar gum, alginic acid, bentonite, carbomers, sodium or calcium carboxymethylcellulose, hydroxyethylcellulose, hydroxypropylcellulose, hydroxypropylmethylcellulose, hydroxypropyl alginate, microcrystalline or powdered cellulose, anhydrous colloidal silica, dextrins, gelatins, kaolin, magnesium aluminum silicate, maltitol, povidone, sorbitan esters, or tragacanth, among others, and mixtures thereof; stabilizing agents such as guar gum, xanthan gum, alginic acid, ascorbic acid, calcium stearate, sodium carboxymethylcellulose, calcium carboxymethylcellulose, ethylcellulose, lecithin, monoethanolamine, potassium chloride, povidone, sorbitol, or xylitol, among others, and mixtures thereof; flavourings such as maltol, vanillin, ethyl vanillin, menthol, citric acid, fumaric acid, ethyl maltol, tartaric acid, mint, artificial or natural fruit flavours, among others, and mixtures thereof; sweeteners such as sorbitol, maltitol, mannitol, dextrose, maltose, xylitol, saccharin, sucrose, sucralose, aspartame, acesulfame potassium, or trehalose, among others, and mixtures thereof; colorants such as curcumin, lactoflavin, iron oxides (red, yellow or black), caramel, lactoflavin phosphate, cochineal red, titanium dioxide, or carotenes, among others, and mixtures thereof; or mixtures of the above.

Preferably, the casein hydrolysate pharmaceutical composition for oral administration is in the form of powder or granules. More preferably, it is in powder form.

The powder is usually prepared by mixing the casein hydrolysate in powder form with at least one pharmaceutically acceptable excipient, generally selected from sweeteners, flavouring agents, colorants, and mixtures thereof. Preferably, the composition in powder form contains an amount of the casein hydrolysate comprised between 70% and 99%, and more preferably comprised between 80% and 95%, expressed as weight percentage relative to the total weight of the composition.

The granulate consists of powder particles that have been aggregated to form larger particles, and are prepared according to procedures that are well known to those skilled in the art, such as dry or wet granulation.

The compositions in powder or granule form are usually taken after dissolution or dispersion in water or another liquid.

The powdered or granulated composition can be presented, for example, in a bulk container such as, for example, a wide-mouth glass jar, so that the necessary dose for each administration is taken each time, preferably, with the help of a measure or dispenser to measure the dose to be administered, or, preferably, it can be presented in the form of single-dose envelopes, which already contain the appropriate unit dose for oral administration. Said envelopes can be made of paper or laminated with aluminum or plastic. Preferably, the unit oral dose comprises between 1 and 30 g of the casein hydrolysate, more preferably between 3 and 15 g, and still more preferably between 4 and 10 g. A particularly preferred dose is about 6 g of hydrolysate.

Some examples are provided below by way of illustration, although not limiting the invention.

### EXAMPLES

### Example 1 Preparation of a Casein Hydrolysate Composition for Oral Administration in Powder Form

A composition in powder form was prepared using the following components:

TABLE 1

| | Weight | % |
|---|---|---|
| Ingredient | (g/unit dose) | (weight) |
| Casein hydrolysate | 6 | 91.58 |
| Sucralose | 0.050 | 0.76 |
| Lemon flavour | 0.50 | 7.63 |
| Colorant | 0.002 | 0.03 |

All the ingredients were mixed well until a homogeneous mixture was obtained and the mixture was introduced into a single-dose sachet.

Each single-dose sachet contained 6 g of casein hydrolysate.

### Example 2 Preparation of a Cream Containing 5% Imiquimod and 15% Casein Hydrolysate

A composition in the form of cream was prepared using the following components:

TABLE 2

| Ingredients | % (weight) |
|---|---|
| Oil phase | |
| Imiquimod | 5.0 |
| Casein hydrolysate | 15.0 |
| Isostearic acid | 5.0 |
| Petrolatum | 10.0 |
| Benzyl alcohol | 2.0 |
| Cetyl alcohol | 2.0 |
| Stearyl alcohol | 2.5 |
| Polysorbate 60 | 2.5 |
| Xanthan gum | 0.5 |
| Sorbitan stearate | 0.5 |
| Aqueous phase | |
| Glycerin | 2.0 |
| Methylparaben | 0.2 |
| Ethylparaben | 0.02 |
| Purified water | q.s. 100% |

To prepare the composition, on the one hand, the ingredients of the oily phase were mixed, heating to about 75° C. On the other hand, the ingredients of the aqueous phase were mixed, under constant stirring and also heating to about 75° C. The aqueous phase was poured over the oily phase, and the mixture was emulsified with an Ultra-Turrax homogenizer at 8000 rpm for about 5 minutes and then the emulsion was stirred in a paddle mixer at 400 rpm for about minutes, and the obtained cream was allowed to cool to room temperature.

### Example 3 Combined Use of Topical Imiquimod and Oral Casein Hydrolysate for the Treatment of Condyloma

The efficacy of the combination of imiquimod and a casein hydrolysate, according to the characteristics defined in the present invention, for the treatment of patients with condyloma was evaluated. Specifically, it was evaluated whether oral supplementation with the casein hydrolysate in patients treated with topical 5% imiquimod produced an improvement in treatment efficacy (rate of complete resolution of condyloma) and/or reduced recurrences (rate of recurrences) after the treatment.

A controlled, observational clinical trial was carried out, which included 44 female patients suffering from condyloma, who were divided into two groups:
Imiquimod group (I) (24 patients): treated with 5% imiquimod cream (3 times per week for up to 16 weeks).
Imiquimod+casein hydrolysate group (I+C) (20 patients): treated with 5% imiquimod cream (3 times per week for up to 16 weeks) plus oral casein hydrolysate (6 g once per day, for 6 months).

Likewise, a controlled, observational clinical trial was carried out with 20 patients suffering from condyloma (Group casein hydrolysate (C)) who were treated only with oral casein hydrolysate for 6 months (6 grams, once per day).

A commercially available casein hydrolysate (HuPaVir®) and a commercially available 5% imiquimod cream (Aldara® 5% cream) were used in the study.

Table 3 shows the the comparative efficacy of the three treatments. In particular, the evaluation of patients at 4, 8, 12 and 16 weeks after starting treatment is included, as well as after 6 months (for group C some points are not available), wherein the percentage of patients with total resolution of condyloma during treatment (up to 16 weeks) and the recurrence rate 6 months after starting the treatment is reported.

TABLE 3

| | Total resolution rate of condyloma (%) | | | | Recurrence rate (%) |
|---|---|---|---|---|---|
| Group | 4 weeks | 8 weeks | 12 weeks | 16 weeks | 6 months |
| I | 8.33 | 28.57 | 58.33 | 72.72 | 14.3 |
| C | nd | nd | 50.00 | 55.00 | 0 |
| I + C | 30 | 68.42 | 100.00 | 100.00 | 0 |

The results show that neither the treatment with imiquimod alone nor the treatment with casein hydrolysate alone are capable of achieving a 100% rate of condyloma resolution. In contrast, with the combined treatment according to the present invention, a complete resolution of condyloma is achieved in all (100%) of the treated patients already at 12 weeks of treatment. Furthermore, no recurrence was observed after 6 months.

1. A method for the treatment of condyloma comprising the administration of a combination of imiquimod and a casein hydrolysate, wherein the hydrolysate comprises peptides, and wherein the molar fraction of peptides carrying a carboxy terminal proline, expressed in %, is more than two times the molar fraction of proline, expressed in %, in the casein substrate used to generate the hydrolysate.

2. The method according to claim 1, wherein the molar fraction of peptides in the hydrolysate carrying the carboxy terminal proline, expressed in %, is at least three times the molar fraction of proline, expressed in %, in the casein substrate used to generate the hydrolysate.

3. The method according to claim 1, wherein the average length of the peptides in the casein hydrolysate is between 3 and 9 amino acids.

4. The method according to claim 1, wherein the molar fraction of peptides carrying the carboxy terminal proline in the casein hydrolysate is at least 25%.

5. The method according to claim 4, wherein the molar fraction of peptides carrying the carboxy terminal proline in the casein hydrolysate is between 30% and 70%.

6. The method according to claim 1, wherein the casein hydrolysate has the following amino acid composition: between 54-64 of lysine, between 22-30 of methionine, between 25-33 of threonine, between 18-26 of histidine, between 26-34 of arginine, between 46-56 of valine, between 34-42 of isoleucine, between 70-80 of leucine, between 33-41 of phenylalanine, between 45-55 of the sum of aspartic acid and asparagine, between 160-180 of the sum of glutamine and glutamic acid, between 20-26 of alanine, between 80-90 of proline, between 40-48 of tyrosine, between 37-45 of serine, between 11-17 of glycine, between 0.8-1.2 of cysteine, and between 6-10 of tryptophan, wherein the amounts are expressed in grams of each amino acid per kilogram of hydrolysate.

7. The method according to claim 1, wherein the imiquimod is administered topically.

8. The method according to claim 7, wherein the imiquimod is administered in the form of a pharmaceutical composition for topical use, with an imiquimod content between 2% and 8%, preferably between 3% and 5%, and more preferably of about 5%.

9. The method according to claim 8, wherein the imiquimod is administered according to a posology selected from once per day, every other day, 1 time per week, 2 times per week, 3 times per week, 4 times per week, and 5 times per week, preferably it is administered 3 times per week.

10. The method according to claim 7, wherein the casein hydrolysate is administered orally.

11. The method according to claim 7, wherein the casein hydrolysate is administered topically and/or orally.

12. The method according to claim 10, wherein the oral casein hydrolysate is administered according to a daily dose between 1 g and 30 g.

13. The method according to claim 12, wherein the daily oral dose of the casein hydrolysate is between 3 g and 15 g.

14. The method according to claim 13, wherein a daily oral dose of about 6 g of the casein hydrolysate is administered in one single administration.

15. The method according to claim 11, wherein a single combined topical formulation is used comprising the imiquimod and the casein hydrolysate.

16. The method according to claim 1, wherein the combination is administered for a maximum period of about 6 months for each episode of condyloma treated.

17. The method according to claim 16, wherein once the combined treatment has ended, the casein hydrolysate is administered on its own for an additional period of time.

18. A pharmaceutical composition for topical administration comprising imiquimod and a casein hydrolysate as active ingredients and at least one pharmaceutically acceptable excipient, wherein the hydrolysate comprises peptides, and wherein the molar fraction of peptides carrying a carboxy terminal proline, expressed in %, is more than two times the molar fraction of proline, expressed in %, in the casein substrate used to generate the hydrolysate.

19. The pharmaceutical composition according to claim 18, wherein the composition is selected from cream, gel, ointment and paste, preferably is selected from cream and ointment.

20. The pharmaceutical composition according to claim 18, wherein the composition comprises between 2% and 8% of imiquimod, preferably between 3% and 5%, and more preferably about 5% of imiquimod, and between 5% and 25% of casein hydrolysate, more preferably between 7% and 15%.
